(19) United States
(12) Patent Application Publication
(10) Pub. No.: US 2005/0004642 A1
(43) Pub. Date: Jan. 6, 2005

(54) Implantable medical lead including overlay

(75) Inventor: Bret R. Shoberg, Hanover, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/897,662

(22) Filed: Jul. 22, 2004

Related U.S. Application Data
(63) Continuation-in-part of application No. 09/854,999, filed on May 14, 2001, now Pat. No. 6,801,809.
Application No. 09/854,999 is a division of application No. 09/507,960, filed on Feb. 22, 2000, now Pat. No. 6,256,542.
Application No. 09/507,960 is a continuation of application No. 09/188,859, filed on Nov. 9, 1998, now Pat. No. 6,052,625.

(51) Int. Cl. A61N 1/05

(52) U.S. Cl. 607/122

(57) ABSTRACT

A medical electrical lead includes a first elongate plastic tube including an outer surface, a conductor extending within the first plastic tube, an electrode having an outer diameter, mounted exterior to the outer surface of the first tube and coupled to a distal portion of the conductor, and a second elongate plastic tube formed of a base polymer to which surface modifying end groups are attached. The second tube is mounted exterior to the outer surface of the first tube, extends from a point adjacent to the first electrode proximally, and has an outer diameter approximately equal to the outer diameter of the first electrode.

### RELATED APPLICATIONS

This application is a continuation-in-part of application Ser. No. 09/854,999 (Attorney docket P-7793.06), which is a divisional of U.S. Pat. No. 6,256,542, filed on Feb. 22, 2002, which is a continuation of U.S. Pat. No. 6,052,625, filed on Nov. 9, 1998, all of which are incorporated by reference herein in there entireties.

### BACKGROUND

This invention relates generally to medical leads and more particularly to implantable medical electrical leads.

In the context of implantable medical electrical leads, it has been found that discontinuities in a lead body's profile, create by electrodes mounted thereabout, can complicate lead implantation and/or extraction in some cases. Furthermore, modified surface properties of a medical electrical lead can enhance implant and/or explant characteristics of the lead.

### BRIEF DESCRIPTION OF THE DRAWINGS

The following drawings are illustrative of particular embodiments of the invention and therefore do not limit its scope, but are presented to assist in providing a proper understanding of the invention. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a plan view of a lead according to the present invention, provided with two coil electrodes;

FIG. 2 is a sectional view through the distal portion of the lead illustrated in FIG. 1, illustrating the construction of the tip-ring assembly;

FIG. 3 is a cross-sectional view of the lead of FIG. 1, taken between the coil electrodes mounted thereon;

FIG. 4 is a sectional view through the ring-coil spacer component illustrated in FIG. 2;

FIG. 5 is a plan view of the distal end of the ring-coil spacer component;

FIG. 6 is a plan view of the proximal end of the ring-coil spacer component;

FIG. 7 is a sectional view through the tip-ring spacer component illustrated in FIG. 2;

FIG. 8 is a plan view of the proximal end of the tip-ring spacer component;

FIG. 9 is a sectional view of the lead of FIG. 1 in the vicinity of one of the coil defibrillation electrodes.

FIG. 10 is a cutaway view of a portion of the lead of FIG. 1 adjacent the connector assemblies;

FIG. 11 is a sectional view through a portion of one of the connector assemblies of the lead of FIG. 1; and

FIG. 12 is a plan view with cutaway sections of a lead according to an alternate embodiment of the present invention.

### DETAILED DESCRIPTION

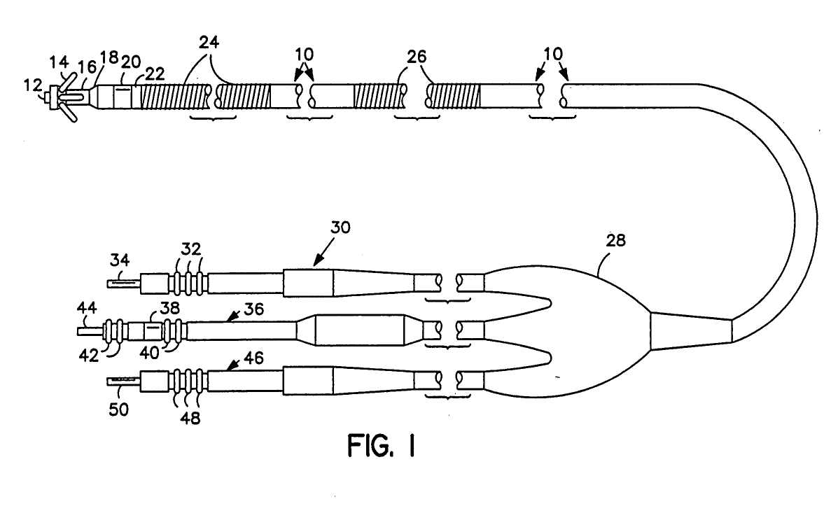

FIG. 1 is a plan view of a lead according to the present invention, embodied as a transvenous cardiac defibrillation lead. The lead is provided with an elongated lead body 10 which carries four mutually insulated conductors therein, not visible in this view. Three of the insulated conductors are stranded or cabled conductors, each coupled to one of ring electrode 20, distal coil electrode 24 and proximal coil electrode 26. A fourth, coiled conductor is coupled to distal or tip electrode 12. The distal portion of the lead includes the tip-ring assembly which includes the tip or distal electrode 12, the tine sheath 16 carrying tines 14, the tip-ring spacer component 18, the ring electrode 20 and the ring-coil spacer component 22. These components together provide a generally rigid assembly, with the tine sleeve 16 fabricated of silicone rubber or relatively softer polyurethanes, and the tip-ring and ring-tip spacers 18 and 22 are fabricated of relatively harder plastics, for example polyurethane having a Shore hardness of at least 75D, to provide a relatively rigid assembly extending to the distal end of distal defibrillation electrode 24.

At the proximal end of the lead body are three connector assemblies 30, 36 and 46, extending from a molded trifurcation sleeve 28, typically formed of silicone rubber. Connector assembly 30 carries a single connector pin 34, coupled to the conductor coupled to the distal coil electrode 24, and is provided with sealing rings 32 to seal the connector assembly 30 within the connector bore of an associated implantable cardioverter/defibrillator. Likewise, connector assembly 46 is provided with a single connector pin 50 coupled to the conductor coupled to the proximal coil electrode 26, and is provided with sealing rings 48. Connector assembly 36 takes the form of an IS-1 type connector assembly provided with a connector pin 44 coupled to the coiled conductor extending to tip electrode 12 and a connector ring 38 coupled to a cabled conductor extending to ring electrode 20. Sealing rings 40 and 42 seal the connector assembly within the connector bore of an associated cardioverter/defibrillator and seal between connector pin 44 and connector ring 38. The lead body 10 which extends from the trifurcation sleeve 28 to the tip-ring assembly at the distal end of the lead is preferably formed of an extruded multi-lumen tube, formed of a plastic substantially less rigid than the ring-tip and tip-ring spacer components 18 and 22. Lead body 10 may for example be formed of silicone rubber and/or a relatively softer implantable polyurethane such as those typically employed in transvenous cardiac lead bodies. In the areas between coil electrodes 24 and 26 and in the area between coil electrode 26 and trifurcation sleeve 28, the lead body is provided with an overlay tubing having essentially the same outer diameter as coil electrodes 24 and 26, which may also be fabricated of silicone rubber, polyurethane or the like.

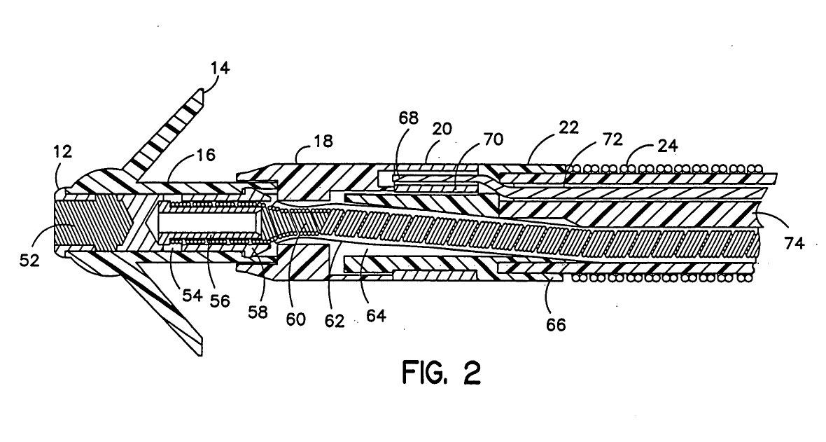

FIG. 2 is a sectional view through the tip ring assembly of the lead of FIG. 1. At the distal end of the assembly is the distal or tip electrode 12 which is provided with an elongated proximally extending shank around which the tine sleeve 16 is mounted. Electrode 12 may be fabricated of platinum/iridium alloy or other biocompatible metal typically used for cardiac pacing electrodes. The shank portion of electrode 12 contains a proximal facing bore in which a monolithic controlled release device 52 is located, containing an anti-inflammatory steroid such as dexamethasone compounded into a plastic matrix, for example as disclosed in U.S. Pat. No. 4,972,848 issued to DiDomenico or U.S. Pat. No. 4,506,680 issued to Stokes, both incorporated herein by reference in their entireties or as implemented in any of the various commercially available steroid eluting cardiac pacing leads.

The shank portion of the electrode 12 also contains a distally facing bore in which the distal end of coiled conductor 60 is located. The distal end of coiled conductor 60 is maintained within the shank by means of a crimping or swaging core 56, with conductor 60 compressed between the electrode 12 and the crimping or swaging core 56. Cross bores 54 are provided through the distal portion of the shank of the electrode, allowing for verification of proper placement of coiled conductor 60 during crimping. The distal-most portion of the shank of the electrode 12 includes a radially extending, distally facing flange 58 which engages with a corresponding internally directed proximally facing circumferential flange, molded into tine sleeve 16. Tine sleeve 16 is preferably fabricated of silicone rubber or a relatively softer polyurethane, for example having a Shore hardness of 80A.

Tine sleeve 16 is adhesively bonded to the tip-ring spacer component 18, for example using silicone medical adhesive or a polyurethane based adhesive, depending on the material of tine sleeve 16. Component 18 overlaps the proximal end of the shank of electrode 12 and the proximal end of tine sleeve 16. Component 18 is provided with a proximally facing internal lumen into which the portion 66 of the ring-coil spacer 22 is inserted. The tip-ring spacer and ring-coil spacer 18 and 22 together define a circumferential groove with corresponding proximal and distal facing shoulders which retain ring electrode 20, when assembled. Components 18 and 22 are preferably fabricated of a relatively more rigid plastic than the tine sleeve 16, for example of polyurethane having a Shore hardness of 75D.

A length of PTFE tubing 62 is heat shrunk around coiled conductor 60 and at least the distal portion of the outer surface thereof has been treated to render the tubing bondable, for example by etching by means of the process commercially available from Zeus Industrial Products, Inc., Orangeburg, S.C. Alternative surface treatments may also be employed to render the tubing bondable, for example using plasma etching or adhesion promoters as described in U.S. Pat. No. 4,944,088 issued to Doan et al., incorporated herein by reference in its entirety. This tubing 62 extends the over the length of the coiled conductor 60 between electrode 12 and connector assembly 36. After assembly, the unfilled space 64 within the tine sleeve 16 and tip-ring and ring-coil spacers 18 and 20 is backfilled with adhesive, bonding the components to themselves and to the etched PTFE tubing 62 and providing for mechanical interlock of all of these components to provide a generally rigid assembly extending from the distal electrode 12 to the distal coil electrode 24.

In this view it can be seen that the ring electrode 20 is provided with an inwardly extending lug 70 having a longitudinal bore into which the distal end of a stranded or cabled conductor 68 has been inserted and which is maintained therein by means of crimps applied to the lug 70. By this mechanism, and in conjunction with the adhesive and mechanical interconnection of the components of the tip-ring assembly shrink tube 62, tensile forces applied to the proximal end of the lead are transmitted to the tip-ring assembly, facilitating removal of the lead without breakage or partial disassembly of the distal portion of the lead.

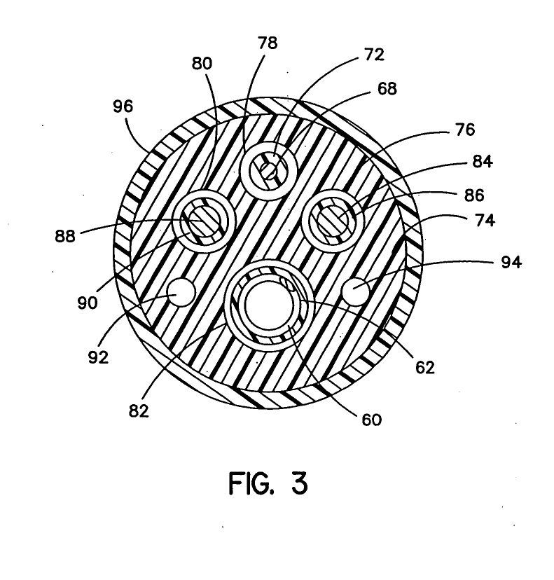

A molded multi-lumen lead body 74 is fabricated of a material softer than the components 18 and 22, for example extruded silicone rubber, or polyurethane having a Shore, for example of 80A or 90A, or the like. Lead body 74 is inserted into a proximal facing recess within ring-coil spacer component 22, and is bonded adhesively therein, for example using a polyurethane or silicone based adhesive. The configuration of the lead body in cross-section is illustrated in more detail in FIG. 3. Coiled conductor 60 and cabled conductor 68 each extend proximal to the connector assembly through lumens in extruded lead body 74. Cabled conductor 68 as illustrated is provided with an ETFE coating 72 which is in turn bonded to the interior of the lumen of extruded lead body 74 in which it is located. At least the distal outer surface of insulation 72 is treated to render it bondable, for example using any of the mechanisms discussed above. Alternatively, the coating 72 may be ETFE which has been modified by exposure to gas plasma, for example using an apparatus as described in U.S. Pat. No. 5,364,662 issued to DiDomenico et al, also incorporated herein by reference in its entirety, with silane used as the feed gas, and ETFE as the plastic to be surface treated.

Coil electrode 24 in this view is visible as having essentially the same outer diameter as the proximal portion of the ring-coil spacer component 22, whereby an essentially isodiametric profile is maintained from the tip-ring spacer up to and including the coil electrode 24. As will be discussed further, this isodiametric profile is maintained proximal to the illustrated portion of the lead by means of overlay tubing, mounted between the electrode coils and between the proximal electrode coil and the trifurcation sleeve (not illustrated in this Figure).

FIG. 3 illustrates a cross-section through the body of the lead of FIG. 1 in an area intermediate the proximal and distal electrode coils. The lead body 74 is visible in cross-section, and is provided with the total of six lumens extending therethrough, including a first lumen 82 in which the coiled conductor 60, coupled to tip electrode 12 (FIG. 2) is located. The PTFE tubing 62 surrounding coiled conductor 60 is also visible in this view. In second and third lumens 76 and 80 are located stranded conductors 84 and 88, each provided with an insulative coating, 86, 90 of ETFE. Conductors 84 and 88 couple the proximal and distal coil electrodes 24 and 26 (FIG. 1) to their associated connector pins at the proximal end of the lead. A fourth lumen 78 carries stranded or cabled conductor 68 which is coupled to ring electrode 20 (FIG. 2). PTFE coating 72, rendered bondable as discussed above by treatment with silane gas plasma or otherwise, is also visible surrounding stranded conductor 68. Compression lumens 92 and 94 are provided to enhance the ability of the lead to resist crush as described in the above cited Shoberg et al patent, and are located diametrically opposite lumens 80 and 76.

Stranded conductors 84, 88, and 68 may correspond to those described in the Shoberg et al., Williams et al. and/or Laske et al. patents cited above. The number and configuration of the individual strands within the conductor may vary as a function of the expected level of current to be carried by the conductors and as a function of the material of which they are fabricated. Typically, it is expected that in the context of a pacing/cardioversion/defibrillation lead, the conductors be fabricated of MP35N alloy wire or silver cored MP35N wire. Coiled conductor 60 may be a monofilar or multifilar coiled conductor, for example having one through five filars, and corresponds to commonly employed coiled conductors used in implantable pacing leads. The coiled conductor 60 may be also fabricated of MP35N alloy or silver cored MP35N wire.

Surrounding the outer periphery of the lead body 74 is overlay tubing 96, which has approximately the same outer diameter and the same thickness as the wire from which the coil electrodes 24 and 26 are fabricated, providing for an essentially isodiametric assembly extending from the proximal coil electrode 26 to the tip-ring assembly illustrated in FIG. 2. A corresponding second overlay tubing extends around lead body 74 proximal to coil electrode 26 (not visible in this view).

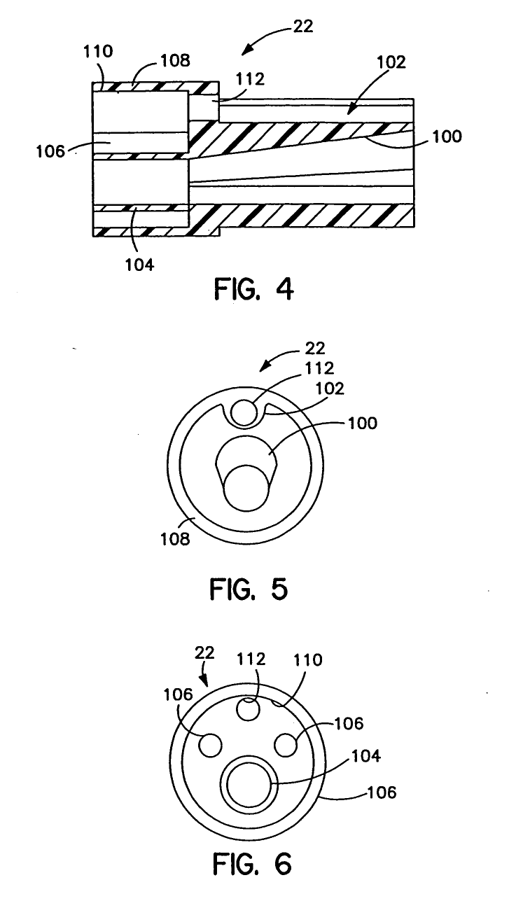

FIG. 4 is a cutaway view through the ring-coil spacer 22. The orientation of the component in this figure is reversed from that in FIG. 2. The ring-coil spacer component 22 is provided with a through lumen 100, through which the coiled conductor 60 (FIG. 2). The component is additionally provided with a generally V-shaped groove 102 in which the lug 70 of the ring electrode 20 (FIG. 2) is located. The proximally facing end of the component 22 is provided with a recess 110 which receives the distal portion of the lead body 74 (FIG. 2). The recess 110 is surrounded by a circumferential wall 108 which has an outer diameter isodiametric to that of the distal coil electrode 24. Extending proximally within recess 110 is a cylindrical sleeve 104 which is inserted into lumen 82 of lead body 74 (FIG. 3). Two proximally extending pins 106 are also located within recess 110 and are configured to be inserted into lumens 80 and 76 of lead body 74 (FIG. 3). Bore 112 allows for passage of the stranded or cabled conductor 68 from the lead body 74 into the lug of the ring electrode 20 (FIG. 2). FIG. 5 is a plan view of the distal end of the ring-coil spacer 22, illustrating the relationship of the U-shaped groove 102, the bore 112, the through lumen 100 and the circumferential wall 108 in more detail. FIG. 6 is a plan view of the proximal end of the component 22, illustrating the relative locations of the circumferential wall 106, the cylindrical sleeve 104, pins 106 and bore 108, in more detail.

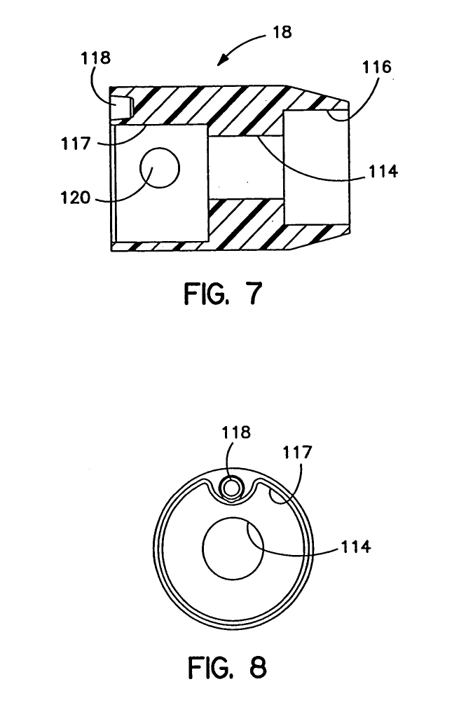

FIG. 7 is a sectional view through the tip-ring spacer 18. Again, the orientation of this view is reversed from that illustrated in FIG. 2. The tip-ring spacer 18 is provided with a through lumen 114, through which the coiled conductor 60 (FIG. 2) extends. A distal-facing recess 116 receives the proximal end of the tine sleeve 16 (FIG. 2) and overlaps the proximal portion of the shank of electrode 12 (FIG. 2). A proximal facing recess 117 receives the distal portion of component 22 as illustrated in FIGS. 4-6. A small proximally facing lumen 118 is provided, which as assembled is aligned with the bore through the lug 70 of ring electrode 20 (FIG. 2), providing a recess into which the cabled or stranded conductor 68 may extend. Bores 120 are provided through the sidewall of component 18, allowing for backfilling of the recess 64 internal to the tip-ring assembly, as illustrated in FIG. 2. FIG. 8 is a plan view of the proximal end of component 18 and illustrates the configuration of the recess 117 which receives the distal portion of component 22, through lumen 114 and recess 118 in more detail.

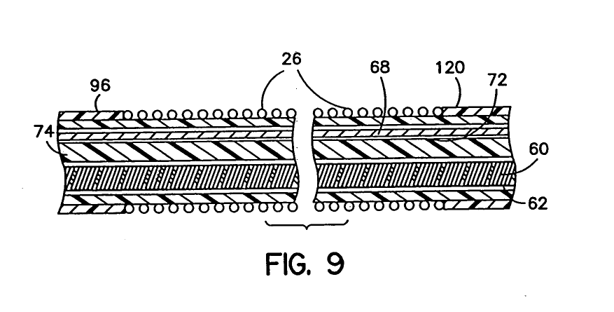

FIG. 9 is a sectional view through the lead of FIG. 1 in the vicinity of the proximal coil electrode 26. Coil electrode 26 is shown located around lead body 74, flanked on its proximal and distal ends by overlay tubing 96 and 120. Overlay tubing 96 corresponds to the same element illustrated in FIG. 3 and extends between coil electrodes 24 and 26. Overlay tubing 120 extends to the trifurcation sleeve 28, illustrated in FIG. 1. Together the coil electrodes 24 and 26 in conjunction with the overlay tubing 96 and 120 provide an essentially isodiametric lead body extending from the trifurcation sleeve to the tip-ring assembly illustrated in FIG. 2. Also visible in this view are stranded or cabled conductor 68 and associated insulative coating 72 and coiled conductor 60 and associated heat shrink PTFE tubing 62. Although not illustrated in FIG. 9 it should be understood that the coil electrodes 24 and 26 may be coupled to stranded or cabled conductors 88 and 84 (FIG. 3) by means of cross-groove crimp sleeves of the sort described in the above cited patent issued to Boser et al.

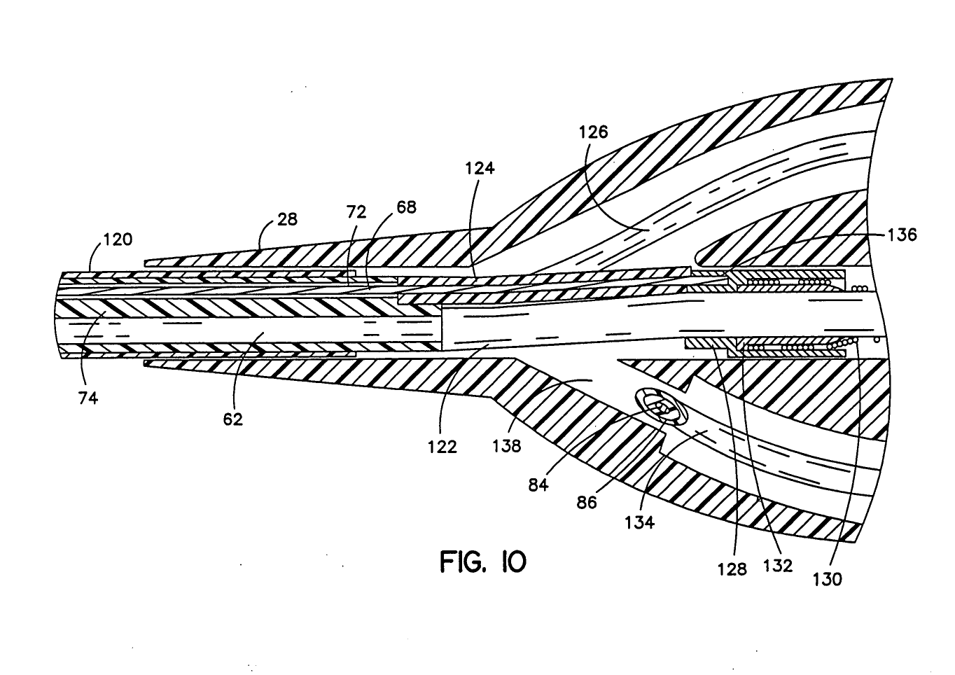

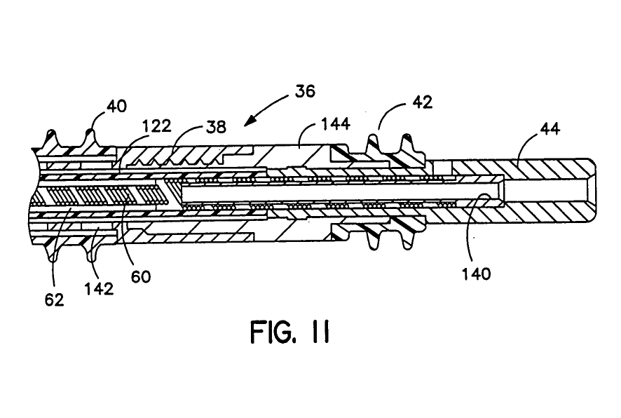

FIG. 10 illustrates a cutaway view through the lead of FIG. 1 in the vicinity of trifurcation sleeve 28. Lead body 74 enters the distal end of trifurcation sleeve 28 and terminates therein. Stranded or cabled conductor 68 extends through lead body 74, out its proximal end and through spacer tubing 124 which extends to transition flange 128, which in turn contains a bore 136 in which the proximal end of cabled or stranded conductor 68 is crimped. At least the proximal outer surface of ETFE insulative coating 72 applied to conductor 68 is made bondable using one of the methods discussed above and as adhesively bonded to the lumen of lead body 74, in the area adjacent to the point at which it exits lead body 74. This adhesive bond provides for a mechanical interconnection between the conductor 68 and the lead body 74, in region of the trifurcation sleeve, which in turn enhances the ability to transmit tensile force provided by the mechanical and electrical interconnection of the stranded or cabled conductor 68 to transition sleeve 128. A coiled conductor 130 is coupled to transition sleeve 128 by means of a crimping or swaging core 132. Connector 130 extends proximally to the IS-1 connector assembly 36 (FIG. 1) where it is coupled to connector ring 38 in a conventional fashion.

Also visible in this view is PTFE shrink tubing 62 which surrounds the coiled conductor 60 (FIG. 2). Shrink tubing 62 and conductor 60 extend proximally inside inner tubing 122 which also extends proximally to the IS-1 connector assembly 36. As discussed below, PTFE shrink tubing 62 is adhesively bonded to the interior of inner tubing 122, in the vicinity of IS-1 connector 36, further enhancing the ability of the lad to transmit tensile force from the proximal to the distal tip of the lead. Also visible in this view are two insulative tubes 126 and 134, each of which surrounds one of the stranded conductors coupled to a coil electrode, and which extend back to the connector assemblies 30 and 46, illustrated in FIG. 1. Tube 134, for example, carries conductor 84 and associated insulative coating 86. The recess 138 defined within trifurcation sleeve 28 is backfilled with silicone rubber medical adhesive, providing a mechanical interconnection of all the components therein. This mechanical interconnection also assists in mechanically coupling the proximal end of the lead body to IS-1 connector assembly 36 and trifurcation sleeve 28.

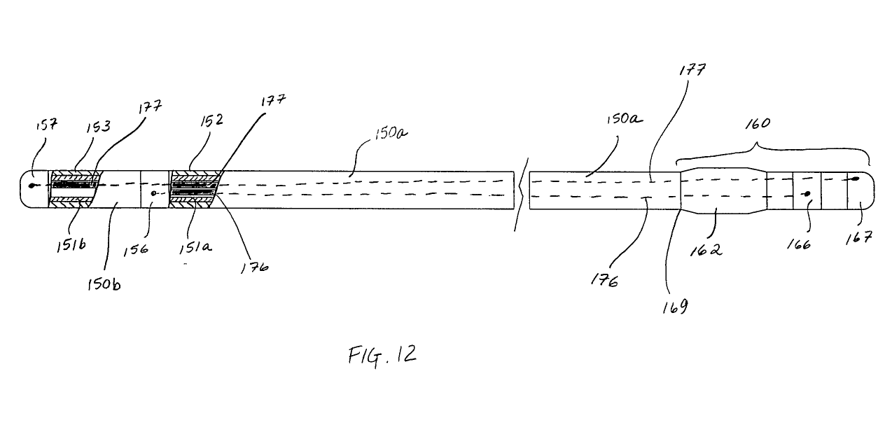

FIG. 11 is a cutaway view through IS-1 connector assembly 36, illustrating the interconnection of the various components including the connector ring 38, connector pin 44 and sealing rings 40 and 42. As illustrated, coiled conductor 60 is coupled to connector pin 44 by means of a crimping or swaging core 140. Coiled conductor 60 and its associated PTFE shrink tubing 62 are located within inner tubing 122, which extends proximally from trifurcation sleeve 28, as illustrated in FIG. 10. Ring electrode 38 is provided with cross bores 142 which facilitate backfilling of the recess between the ring electrode 38 and the inner tubing 122, serving to mechanically interconnect the inner tubing 122 to ring electrode 38 and sealing rings 40. Ring electrode 38 is in turn mechanically interconnected with connector pin 44 by means of injection molded spacer 144, fabricated according to U.S. Pat. No. 4,572,605 issued to Hess, incorporated herein by reference in its entirety. At least the proximal outer surface of PTFE shrink tubing 62 applied to conductor 60 is made bondable using one of the methods discussed above and is adhesively bonded to the lumen of inner tubing 122, further facilitating transmission of tensile forces from the proximal end to the distal end of the lead body, as discussed above.

FIG. 12 is a plan view with cutaway sections of a lead according to an alternate embodiment of the present invention. FIG. 12 illustrates the lead including a lead body 150a terminated at a proximal end by a connector 160 and formed of a first elongate plastic tube 151a and a second elongate plastic tube or overlay tube 152 mounted exterior to an outer surface of the first tube 151a; a first electrode 156, mounted exterior to the first tube 151a, is coupled to a first connector contact 166 via a first insulated conductor 176 extending within lead body 150a. According to embodiments of the present invention, second tube 152 extends proximally from a point adjacent to first electrode 156 and has an outer diameter approximately equal to an outer diameter of first electrode 156; second tube 152 may extend to a distal end 169 of connector 160, which is illustrated herein coupled to lead body 150a by a connector sleeve 162, or may extend to a point in the vicinity of distal end 169.

FIG. 12 further illustrates a lead body distal extension 150b extending distally from first electrode 156 toward second electrode 157 and being formed of a first plastic tube extension 151b and a third plastic tube or overlay tube 153 mounted exterior to an outer surface of first plastic tube extension 151b; a second insulated conductor 177 couples second electrode 157 to a second connector contact 167. According to further embodiments, third tube 153 extends distally from a point adjacent first electrode 156 to a point adjacent second electrode 157 and has an outer diameter approximately equal to an outer diameter of second electrode 157. The outer diameter of second electrode 157 may be approximately equal to or less than the outer diameter of first electrode 156.

Overlay tubes 152 and 153 may have a snug or loose fit about first tube 151a and first tube extension 151b, respectively, and are preferably bonded to tubes 151a and 151b in proximity to electrodes 156 and 157. Overlay tube 152 may be further secured about first tube 151a by extending beneath sleeve 162, similar to the configuration illustrated in FIG. 10.

Electrodes 157 and 156 may be of any form known to those skilled in the art accommodating low voltage stimulation, or high voltage stimulation, or sensing, or any combination of these. According to one example electrodes 157 and 156 are tip and ring electrodes, respectively, forming a pace/sense pair, while according to another example, electrode 156 is further adapted for high voltage stimulation. Tube 151a may have a single lumen as illustrated in FIG. 12 or may have multiple lumens, for example similar to that illustrated in FIG. 3. Although first and second conductors 176 and 177 are each insulated, tubes 151a and 151b serve as a primary insulation to electrically isolate conductors 176 and 177 from an implant environment. According to an alternate embodiment that includes a single insulated conductor, the insulation of that conductor forms the primary insulation about which an overlay tube is mounted without an intervening tube.

According to yet another set of embodiments of the present invention, second tube 152, and, in some cases, third tube 153, is formed of a base polymer to which surface modifying end groups (SME's) are attached; a general description of such polymeric compositions may be found in U.S. Pat. No. 5,589,563 to Ward et al., which is incorporated by reference in its entirety herein. It should be noted that previously illustrated overlay tubes, for example tube 96 of FIG. 3 and tube 120 of FIGS. 9 and 10, are formed of such polymeric compositions, according to further embodiments of the present invention. Thus, without modifying a primary insulation, surface properties of lead bodies may be tailored via overlay tubes. Examples of desirable surface properties include but are not limited to lubricity and enhanced biocompatibility.

Suitable base polymers for overlay tubes having SME's include, but are not limited to, polyurethanes, silicones, polyurethane-silicone hybrids, polyimides, fluoropolymers and polyolefins, and suitable SME's include, but are not limited to, fluorocarbons, silicones and polyethylene oxides (PEO). According to one embodiment an overlay tube is formed of a polyurethane base polymer including silicone SME's; such a combination may prevent environment stress cracking of the overlay tube that sometimes occurs with implanted polyurethanes.

Some exemplary embodiments wherein SME's enhance biocompatibility are described as follows:

An overlay tube is formed from a polyurethane base polymer having branched PEO molecules as SME's to which a biologically active agent is attached; co-pending and commonly assigned U.S. patent application 20030204230 describes such a material in detail and is incorporated by reference herein in its entirety.

An overlay tube is formed from a polyurethane base polymer having fluorocarbon molecules as SME's; co-pending and commonly assigned U.S. patent application 20030028224 describes such a material in detail and is incorporated by reference herein in its entirety.

Variations of the invention, using one or more of the features enumerated herein, may of course be used in conjunction with leads having a greater or fewer number of electrodes and conductors. As such, the above disclosure should be considered exemplary, rather than limiting, with regard to the claims which follow.

1. A medical electrical lead comprising:
a first elongate plastic tube including a proximal end, a distal end and an outer surface;
a first conductor extending within the first plastic tube from the proximal end toward the distal end;
a first electrode having an outer diameter, mounted exterior to the outer surface of the first tube and coupled to a distal portion of the first conductor;
a connector terminating the proximal end of the first elongate plastic tube including a connector contact coupled to a proximal portion of the first conductor; and
a second elongate plastic tube formed of a base polymer to which surface modifying end groups are attached, the second tube being mounted exterior to the outer surface of the first tube, extending from a point adjacent to the first electrode proximally toward the connector, and having an outer diameter approximately equal to the outer diameter of the first electrode.

2. The lead of claim 1, further comprising:
a second conductor extending within the first plastic tube from the proximal end toward the distal end;
a second electrode having an outer diameter, mounted exterior to the outer surface of the first tube, distal to the first electrode, and coupled to a distal portion of the second conductor; and
a third plastic tube mounted exterior to and around the outer surface of the first tube, extending from a point adjacent to the first electrode distally to a point adjacent to the second electrode, and having an outer diameter approximately equal to the outer diameter of the second electrode.

3. The lead of claim 2, wherein the third elongate plastic tube is formed of a base polymer to which surface modifying end groups are attached.

4. The lead of claim 1, wherein the first electrode is a coil.

5. The lead of claim 2, wherein the first electrode is a coil.

6. The lead of claim 2, wherein the second electrode is a coil.

7. The lead of claim 1, wherein the base polymer is selected from the group consisting of polyurethanes, silicones, polyimides, fluoropolymers and polyolefins.

8. The lead of claim 1, wherein the base polymer is a polyurethane-silicone hybrid.

9. The lead of claim 1, wherein the surface modifying end groups are fluorocarbons.

10. The lead of claim 1, wherein the surface modifying end groups are silicones.

11. The lead of claim 1, wherein the surface modifying end groups are polyethylene oxides.

12. The lead of claim 11, wherein the polyethylene oxides are branched.

13. The lead of claim 11, wherein a biologically active agent is attached to one or more of the polyethylene oxide end groups.
